(19) United States
(12) Patent Application Publication
(10) Pub. No.: US 2014/0322767 A1
(43) Pub. Date: Oct. 30, 2014

(54) DROPLET MICROREACTOR

(71) Applicants: Gilles MARCHAND, Pierre Chatel (FR); Françoise VINET, Grenoble (FR); Guillaume DE LAPIERRE, Vif (FR); Fatima HASSINE, Casablanca (MA); Saïd GMOUH, Rennes (FR); Michel VAULTIER, Chateaugiron (FR)

(72) Inventors: Gilles MARCHAND, Pierre Chatel (FR); Françoise VINET, Grenoble (FR); Guillaume DE LAPIERRE, Vif (FR); Fatima HASSINE, Casablanca (MA); Saïd GMOUH, Rennes (FR); Michel VAULTIER, Chateaugiron (FR)

(21) Appl. No.: 14/270,799

(22) Filed: May 6, 2014

Related U.S. Application Data
(63) Continuation of application No. 11/631,554, filed on Jan. 4, 2007, now abandoned.

(30) Foreign Application Priority Data
Jul. 8, 2004 (FR) 0407623

(51) Int. Cl. C07D 221/18; B01L 3/00; C07C 227/16; C07C 41/14; C12P 17/12

(52) U.S. Cl. CPC C07D 221/18; C07C 41/14; C12P 17/12; C07C 227/16; B01L 3/502792
USPC 435/122; 546/61; 422/547; 568/640; 560/74; 204/450

(57) ABSTRACT

The present invention relates to a droplet microreactor, i.e. a microreactor consisting of a droplet of a specific liquid, the microreactor being wall-less, wherein the interface of the specific liquid with the ambient environment and with the support on which the droplet is deposited defines the limits of the microreactor. The microreactor is characterized in that it consists of a droplet comprising at least one ionic liquid. The present invention also relates to methods for carrying out chemical or biochemical reactions and/or mixes using said droplet microreactor, and also to a lab-on-chip comprising a microreactor according to the invention.

### TECHNICAL FIELD

The present invention relates to a droplet microreactor, i.e. to a microreactor consisting of a droplet of a specific liquid, the microreactor being wall-less, wherein the interface of the specific liquid with the ambient environment and with the support on which the droplet is deposited defines the limits of the microreactor.

The present invention also relates to methods for carrying out chemical or biochemical reactions and/or mixes using said droplet microreactor, and also to a lab-on-chip comprising a microreactor according to the invention.

The specific liquid used in the present invention is an ionic liquid or a mixture of ionic liquids.

The present invention finds numerous applications, in particular in lab-on-chips where very small volumes of reaction media are generally used. It makes it possible, for example, to carry out syntheses on a soluble support, parallel syntheses, convergent syntheses, or immobilizations on the ionic liquids of chemical or biological molecules that may be detected (target molecules), or to detect (probe molecules) enzymatic reactions, catalyst heterogenizations and homogeneous catalysts, method optimizations, dangerous reactions, combinatorial chemistry reactions, etc.

In the description which follows, the references between square brackets [ ] refer to the attached reference listing.

### PRIOR ART

Numerous microsystems or microreactors intended for carrying out chemical or biochemical reactions on a microlitre or even nanolitre scale are described in the literature, for example in documents [1]-[5].

Most of these devices involve a system of channels, as described in documents [1]-[3], included in a packaging, in particular made of glass, metal, silicon, organic polymer, ceramic, etc.

However, these microsystems generate a certain number of problems: the channels become easily blocked, they are subject to load losses in the hydrodynamic mode, for example when syringe-pumps and pumps are used, and it is often difficult to prevent dead volumes and to optimize a low-cost microsystem compatible with an aggressive chemical environment, in particular by virtue of the solvents used, the working temperatures, the pressures, etc.

Other microsystems use droplets of liquids in which the authors carry out reactions. These microsystems are described, for example, in documents [4] and [5]. These droplets of liquids may be aqueous or organic solvents.

However, in both cases, the authors are confronted with the evaporation of these solvents, which, at best, implies the need for a cover or, at worst, makes them impossible to use. In addition, in the case of water, few organic chemistry reactions are known. In the case of organic solvents, the users are confronted with the problem of toxicity, for example by inhalation, of safety, for example due to risks of inflaming, and also with recycling.

Furthermore, in order to carry out chemical reactions in these microsystems, it is often necessary to displace the reagents in order to carry out these reactions or mixes. In the case of channels, the displacement of the reagents is often imposed by means of pumps that make it possible to control pressures, or of syringe-pumps that make it possible to control flow rates.

The displacement can also be carried out by electroosmosis; which requires the control of surface charges.

In droplet systems, electrowetting (EWOD: for “electrowetting on dielectric”) and acoustic waves are generally used, as described, for example, in document [5]. For aqueous solvents, this does not generally pose too many problems, whereas for organic solvents, only some of them are compatible with these techniques. This is because most solvents are insulating, and yet the solvents must be conducting in order to be usable in electrowetting.

However, few chemical reactions are carried out in an aqueous medium, although a certain number of authors work in such media, as illustrated in document [6].

Finally, several chemical applications, such as combinatorial chemistry, in situ syntheses, etc., require the use of soluble supports, for example of polyethylene glycols, or insoluble supports, for example Merrifield-type resin beads, of silica, etc., in the reaction medium, as illustrated by documents [7] and [8]. The existing approaches have several drawbacks:

(i) On insoluble supports:
reactions in a heterogeneous medium have kinetics that are generally slower than in solution;
sometimes certain reactions which are feasible in solution do not function on a solid support; and
the reactions are difficult to monitor.
   (ii) On soluble polymer supports:
the purification of the reaction products is very tricky,
parallelization is difficult,
low specific charge, and
recycling is difficult.

There exists therefore a real need for a reactor that does not have the abovementioned drawbacks of channel microsystems, of microsystems comprising droplets of aqueous or organic solvents, and of the soluble or insoluble supports of the prior art.

### DISCLOSURE OF THE INVENTION

The present invention satisfies precisely this need, and also others, explained below, by providing a microreactor characterized in that it consists of a droplet comprising at least one ionic liquid.

The present invention also satisfies this need, and also others, explained below, by providing, according to a first embodiment, a method for carrying out a chemical or biochemical reaction, comprising the following steps:
depositing a droplet of at least one ionic liquid onto a surface;
introducing, into the at least one ionic liquid, before or after it is deposited in the form of a droplet, at least one chemical or biochemical reagent,
chemically or biochemically reacting, in said droplet, the reagent with the ionic liquid or the reagents with one another.

The present invention also satisfies this need, and also others, explained below, by providing a method of mixing droplets of ionic liquid, comprising the following steps:
depositing a first droplet of at least one first ionic liquid onto a surface;
depositing a second droplet of at least one second ionic liquid onto said surface;
optionally, introducing into the first ionic liquid, before or after it is deposited in the form of a droplet onto the surface, at least a first chemical or biochemical reagent;
optionally, introducing into the second ionic liquid, before or after it is deposited in the form, of a droplet onto the surface, at least a second chemical or biochemical reagent;
bringing the first droplet and the second droplet together so as to form a single droplet.

Thus, the droplets of ionic liquids, which may be identical or different in terms of their volume and/or their content, each comprising or not comprising, independently of one another, one or more reagent(s), and each comprising or not comprising, independently of one another, a solvent, are mixed with one another and so therefore also is their possible content, by bringing together said droplets to form a single droplet.

The step consisting in bringing together the droplets can be followed by a step consisting in chemically or biochemically reacting, in the droplet formed by bringing them together, reagents with one another when they are present in one and/or the other of the droplets, and/or with the first and/or the second ionic liquid(s), in particular when this (these) ionic liquid(s) is (are) functionalized.

The present invention therefore also satisfies, for example, the abovementioned need, and also others disclosed below, by providing, according to a second embodiment, a method for carrying out a chemical or biochemical reaction, comprising the following steps:
depositing a first droplet of at least one first ionic liquid onto a surface;
depositing a second droplet of at least one second ionic liquid onto said surface;
introducing into the first ionic liquid, before or after it is deposited in the form of a droplet onto the surface, at least a first chemical or biochemical reagent;
introducing into the second ionic liquid, before or after it is deposited in the form of a droplet onto the surface, at least a second chemical or biochemical reagent;
bringing the first droplet and the second droplet together on said surface so as to form a single droplet; and
chemically or biochemically reacting, in the droplet formed by the first and second droplets brought together, said first reagent with said second reagent.

The aim of the present invention is to provide a novel use of ionic liquids as a microreactor, more particularly for applications in analytical techniques and chemical and biochemical reactions carried out on lab-on-chips. The present invention therefore also relates to a lab-on-chip comprising at least one microreactor according to the invention.

In fact, the microreactor of the present invention is a wall-less reactor: it is the interface of the ionic liquid with the ambient environment that defines the limits of the microreactor. For this reason, in the present description, it is also called a “droplet microreactor”.

The ionic liquids, on the basis of which the present invention is implemented, have a certain number of advantageous physicochemical properties described in document [9]. These properties are, in particular:
their low volatilities and their very low vapour tensions: the ionic liquids are not very volatile and have very low vapour tensions, unlike volatile organic solvents (VOS) and aqueous solvents, and there is therefore no problem of evaporation when they are used in droplet format. They are in particular less volatile than water and most organic solvents such as, for example, ether, tetrahydrofuran, dichloromethane, chloroform, ethanol, methanol, toluene or acetonitrile, solvents for which the boiling point is less than or equal to 110° C. Above this temperature, they pass into the gaseous state and can no longer be used as solvents in chemistry in conventional reactors. On the other hand, ionic liquids do not exhibit this problem;
their great thermal stability: ionic liquids are very thermally stable, some up to more than 400° C., unlike
their low inflammability,
their high capacity for solubilizing both salts and neutral organic molecules and polymers, and various substances such as complexes of transition metals, for instance catalysts;
they can be readily recycled;
they can be functionalized and can therefore be used as soluble supports with a high specific charge and make it possible to carry out reactions with the same reactivity as in an aqueous or organic solution;
the chemical and biochemical reactions can be monitored therein readily by modern analytical techniques such as nuclear magnetic resonance (NMR) or high performance chromatography techniques (HPLC);
the reaction products can be readily purified.

The following properties of the ionic liquids can also be mentioned, in particular in terms of their advantage in lab-on-chip applications:
these liquids can be used in electrochemistry and have a large electrochemical window; and
they are compatible with biological molecules such as enzymes, proteins, nucleic acids (DNA and RNA), glycoproteins, lipids, etc.

According to the invention, the at least one ionic liquid can be chosen from all appropriate ionic liquids and onium salts known to those skilled in the art, and also from mixtures thereof. Documents [9] and [10] describe examples of ionic liquids, onium salts and mixtures thereof that can be used to implement the present invention, and also their physicochemical properties and the method(s) for producing them.

The ionic liquid that can be used is in liquid form at ambient temperature; it can be represented by the formula A₁⁺X₁⁻, in which A₁⁺ represents a functional or nonfunctional cation or else a mixture of cations in which either none of the cations is functional or at least one of the cations is functional, and in which X₁⁻ is a functional or nonfunctional anion, or a mixture of anions in which either none of the anions is functional or at least one of the anions is functional. The expression “ionic liquid” denotes in general a salt or a mixture of salts of which the melting point is between −100° C. and 250° C.

The term “ionic liquid”, unless otherwise specified, is intended to mean a pure ionic liquid or a mixture of ionic liquids, which may be functionalized or nonfunctionalized, or a mixture of one or more functionalized or nonfunctionalized ionic liquids with one or more reagents and/or solvents.

The term “nonfunctionalized ionic liquid” or “matrix ionic liquid” is intended to mean an ionic liquid capable of solubilizing one or more chemical or biological species such as inorganic or organic salts, organic molecules, or polymers of natural or synthetic origin. The expression “nonfunctionalized ionic liquid” therefore denotes a solvent consisting of an ionic liquid. These new “solvents” are non-volatile and have a very low vapour tension. They are also polar and have the ability to dissolve functionalized onium salts that may therefore be used as soluble supports as described in document [10]. They can be used pure or as a mixture.

The term “functionalized ionic liquid” or “task-specific ionic liquid” or “dedicated ionic liquid” is intended to mean an ionic liquid of formula indicated above, of which either the cation, or the anion, or both, carries or carry a function capable of reacting with a reagent present in the droplet. They can be used pure or as a mixture.

The expression “functional cation” denotes a molecular group that has at least one chemical function, a part of this group carrying a positive charge. The expression “functional anion” denotes a molecular group that has at least one chemical function, a part of this group carrying a negative charge. The expression “nonfunctional cation” denotes a molecular group that does not have a chemical function, a part of this group carrying a positive charge. The expression “nonfunctional anion” denotes a molecular group that does not have a chemical function, a part of this group carrying a negative charge.

When the ionic liquid A₁⁺X₁⁻ comprises no functional ion, it is called a “nonfunctionalized ionic liquid”. It serves as a reaction medium that is inert or a matrix with respect to the reagents, but is capable of dissolving them.

When the ionic liquid A₁⁺X₁⁻ comprises at least one functional ion, it is called a “functionalized ionic liquid”. It can serve, firstly, as a reaction medium and, secondly, as a soluble support or matrix.

In the present invention, said at least one ionic liquid may therefore be a functionalized or nonfunctionalized ionic liquid, but also a mixture of functionalized ionic liquid(s) and nonfunctionalized ionic liquid(s). The droplet of ionic liquid that forms the microreactor can therefore comprise, in addition to the functionalized ionic liquid, a nonfunctionalized ionic liquid, or else, in addition to the nonfunctionalized ionic liquid, a functionalized ionic liquid.

The choice of, or of the mixture of, ionic liquid(s) depends on the mixture and/or on the chemical or biochemical reaction that will be carried out in the “droplet reactor” of the present invention.

The fact of having mixtures of ionic liquids is not a hindrance in the case where all the constituents of the mixture are chemically inert under the conditions of use when this inertia is required in the implementation of the present invention. For example, a mixture of nonfunctional tetraalkylammonium or phosphonium salts can be used. Furthermore, the melting point of a mixture is lower than the melting point of the constituent of the mixture that melts at the lowest temperature. It may therefore be very important to turn to a mixture in order to have an ionic liquid with a reasonable melting temperature.

Some functionalized salts, in particular with large anions such as NTf2⁻, PF₆⁻, BF₄⁻ or CF₃SO₃⁻, may be liquid at ambient temperature or may melt at low temperature, for example

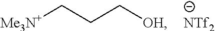

is liquid at ambient temperature. This ionic liquid is prepared by alkylation of Me₃N according to the following reaction:

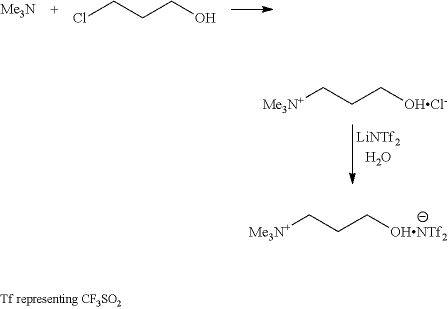

In the present invention, it is possible to use, as A₁⁺, a nonfunctional cation or a mixture of nonfunctional cations, and as X₁⁻, a nonfunctional anion or a mixture of nonfunctional anions.

In the present invention, it is also possible to use, as A₁⁺, a functional cation or a mixture of cations, at least one of which is functional, and/or as X₁⁻, a functional anion or a mixture of anions, at least one of which is functional, said functional cations and functional anions corresponding to an ionic entity, i.e. respectively a cationic or anionic entity, linked to at least one function F₁, F₁ ranging from F₀ to Fₙ, n being an integer ranging from 1 to 10.

The expression “ionic entity” denotes the part of the cation or of the anion that carries the charge, respectively positive or negative.

The function F₁ can in particular be chosen from the following functions: hydroxyl, carboxylic, amide, sulphone, primary amine, secondary amine, aldehyde, ketone, ethenyl, ethynyl, dienyl, ether, epoxide, phosphine (primary, secondary or tertiary), azide, imine, ketene, cumulene, heterocumulene, thiol, thioether, sulphoxide, phosphorus groups, heterocycles; sulphonic acid, silane, functional aryl or stannane, and any function resulting from a chemical, thermal or photochemical conversion, or a conversion by microwave irradiation, of the above functions.

For example, the at least one ionic liquid can be chosen from an imidazolium salt, more generally an ammonium salt, a phosphonium salt, an onium salt or a mixture of these salts. As indicated above, these salts may be functionalized or nonfunctionalized.

By way of examples of ionic liquids serving as a matrix, i.e. of nonfunctionalized ionic liquids, mention may be made of the following:
1-butyl-3-methylimidazolium tetrafluoroborate [bmim][BF₄];
1-butyl-3-methylimidazolium hexafluorophosphate [bmim][PF₆];
1-butyl-3-methylimidazoliumbis(trifluoromethylsulphonyl)imide [bmin][NTf₂];
1-ethyl-3-methylimidazolium hexafluorophosphate [emim][PF₆]; and
butyltrimethylammoniumbis(trifluoromethylsulphonyl)imide [btma][NTf₂].

For implementing the present invention, use may be made, for example, of an ionic liquid as defined above, in a stable composition containing in solution: at least said ionic liquid of formula A₁⁺X₁⁻, playing the role of a liquid matrix, and at least one functionalized ionic liquid (“task-specific”), for example a functionalized onium salt, of formula A₂⁺X₂⁻, as reaction support,

the functionalized onium salt, for example the functionalized ionic liquid, being dissolved in the nonfunctionalized ionic liquid, so as to form a homogeneous phase,
A₁⁺ representing a nonfunctional cation or a mixture of cations in which none of the cations is functional, and X₁⁻ representing a nonfunctional anion or a mixture of anions in which none of the anions is functional,
A₂⁺ representing a functional or nonfunctional cation or a mixture of cations in which none of the cations is functional or in which at least one of the cations is functional, and X₂⁻ representing a functional or nonfunctional anion or a mixture of anions in which none of the anions is functional or in which at least one of the anions is functional,
with the proviso that A₂⁺ and/or X₂⁻ represent(s) or comprise(s) respectively a functional cation and/or a functional anion,
said functional cations and functional anions corresponding to an ionic entity Y—, i.e. respectively cationic Y⁺- or anionic Y⁻—, linked, optionally by means of an arm L, in particular an alkyl group containing from 1 to 20 carbon atoms, to at least one function Fᵢ, Fᵢ ranging from F₀ to Fₙ, n being an integer ranging from 1 to 10, it being possible for the functional cation to be represented in the form Y⁺-L-Fᵢ, and for the functional anion to be represented in the form Y⁻(L)ₖ-Fᵢ, k being equal to 0 or 1, the functional anion possibly representing, when k is equal to 0, a simple anion, corresponding to Y⁻Fᵢ, in particular chosen from: OH⁻, F⁻, CN⁻, RO⁻, RS⁻, RSO₃⁻, RBF₃⁻, where R represents an alkyl group containing from 1 to 20 carbon atoms or an aryl group containing from 6 to 30 carbon atoms.

The expression “stable composition” denotes a homogeneous mixture composed of the liquid matrix A₁⁺X₁⁻ and of the functionalized salt(s) A₂⁺X₂⁻. This composition is said to be stable insofar as it does not undergo any spontaneous conversions over time. It is possible to verify that this composition is stable by spectroscopic analysis by means of nuclear magnetic resonance (NMR), infrared (IR), ultraviolet (UV) in the visible range, mass spectrometry or chromatography methods.

The expression “functionalized ionic liquid” denotes an entity of the type A₂⁺X₂⁻ in which the cation and/or the anion carries a function Fᵢ as defined above. This function confers on said functionalized ionic liquid and on the stable composition, of which it is part, chemical and/or physicochemical properties.

The expression “functionalized onium salt” denotes ammonium, phosphonium and sulphonium salts, and also all the salts resulting from the quaternization of an amine, of a phosphine, of a thioether or of a heterocycle containing one or more of these heteroatoms, and carrying at least one function Fᵢ. This expression also denotes an onium salt of which the cation as defined above is not functionalized, but of which the anion carries a function Fᵢ. This expression can also denote a salt of which the anion and the cation carry a function Fᵢ. A preferred functionalized onium salt is in particular chosen from the following:

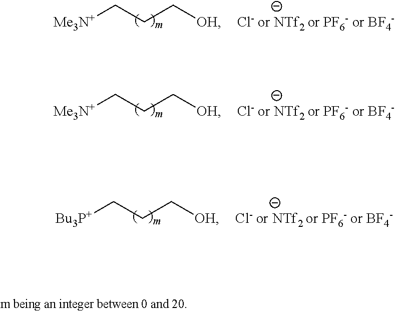

A preferred nonfunctionalized onium salt is in particular chosen from the following: imidazolium, pyridinium, Me₃N⁺-Bu or Bu₃P⁺-Me cations, NTf₂⁻, PF₆⁻ or BF₄⁻ anions.

In the present invention, the ionic liquids can therefore be used pure or else as a mixture. Said mixture may, for example, be a task-specific ionic liquid at a certain concentration in another ionic liquid that acts as a matrix, for example for carrying out supported reactions as described in document [10]. The functional salt dissolved in the matrix may be a liquid or a solid with a high melting point, the important factor being that it is soluble in the matrix. It may also be an ionic liquid dissolved in one or more solvent(s), where appropriate chosen so as to be compatible with the techniques for displacing the droplet(s) when these techniques are implemented in the context of the present invention. A functionalized onium salt that is liquid at a temperature of less than 100° C. may be a task-specific ionic liquid or a solution of a functionalized salt in a nonfunctional ionic liquid matrix.

According to the invention, when the ionic liquid that forms the microreactor comprises at least one solvent, it may be any solvent that can be used for implementing the present invention, preferably compatible with the ionic liquid(s) used, preferably miscible or partially miscible. In the latter case, the solvent is sufficiently miscible to allow the mixing or the chemical reaction in accordance with the present invention to be carried out.

The at least one solvent can be chosen, for example, from organic solvents such as dichloromethane, chloroform, trichloroethylene, dichloromethylene, toluene, acetonitrile, propionitrile, dioxane, N-methylpyrrolidone, tetrahydrofuran (THF), dimethylformamide (DMF), ethyl acetate, ethanol, methanol, heptane, hexane, pentane, petroleum ether, cyclohexane acetone, or isopropanol; or from aqueous solvents such as sulphuric acid, phosphoric acid, sodium hydroxide, etc. This list is not of course limiting, and any solvent compatible with the ionic liquids and with the mixing and/or the chemical reaction carried out can be used for implementing the present invention.

Volatile solvents such as those mentioned above (VOS and above solvents) that are miscible with the ionic liquids can be used. These solvents evaporate, in particular when heating is carried out.

According to the invention, the ionic liquid that forms the microreactor can also comprise at least one reagent. This (these) reagent(s) may, for example, be that (those) used for carrying out, in the droplet microreactor of the present invention, the mixing(s) of reagents and/or the chemical or biochemical reaction(s). It may also involve one or more reagent(s) used for detecting and/or analysing the initial products and/or final products derived from the chemical or biochemical reactions carried out in the microreactor.

The at least one reagent can be introduced into the ionic liquid in the form of a powder (solid), in the form of a liquid or in solution. Whatever the method of implementing the present invention, the introduction of the reagent can be carried out by simply depositing the liquid reagent, in or onto the ionic liquid, before or after the droplet(s) is (are) deposited onto the surface. A homogenization of the ionic liquid/reagent mixture can then be carried out, for example by mixing, or else, when a droplet is involved, for example by means of vibrations or by simple brownian movement.

According to the invention, when the reagent to be introduced into the ionic liquid is volatile, it is advantageously possible to fix it in the microreactor of the present invention by using an ionic liquid specially functionalized so as to fix said reagent. Thus, when the reagent is introduced into the ionic liquid, it is fixed by the latter and can no longer evaporate.

When the reagent is in solution, the solution is preferably realized by means of a solvent that is chemically compatible with the ionic liquid, i.e. that does not chemically react with the ionic liquid and, also preferably, that does not interfere with the chemical or biochemical reaction that has to be carried out in the droplet. The solvent used must, of course, also be at least partially miscible with the ionic liquid. Examples of solvents that can be used to this effect are given above. After the introduction of the reagent in solution into the ionic liquid, the solvent used can remain in the ionic liquid or can be evaporated from the ionic liquid, for example by heating.

According to the invention, when the reagent is in the form of a liquid or in solution, it is also possible to deposit a droplet of this solution of reagent onto the surface in proximity to the droplet of ionic liquid that forms the microreactor of the present invention and to bring these two droplets together to form a single droplet in order to mix their content. The bringing together of these two droplets can be carried out, for example, by one of the displacement techniques described below, for example by electrowetting. Thus, the introduction of the reagent into the microreactor of the present invention can be carried out by coalescence of a droplet of ionic liquid and of a droplet of the reagent on the surface.

In the method of the invention, regardless of the embodiment, the droplet(s) can be deposited onto the surface, for example of a lab-on-chip, by any technique known to those skilled in the art, for example by a technique chosen from the group comprising manual deposition, deposition by means of an automated or nonautomated droplet dispenser, for example from a reservoir of ionic liquid, or else deposition by fractionation of a larger droplet deposited onto the surface.

According to the invention, each droplet that forms a microreactor has a volume such that it forms a droplet. Where appropriate, when the droplet must be displaced, it must be possible for this droplet to be displaced by means of the displacement technique chosen. For example, for a use in a lab-on-chip, in general, the droplet has a volume of 10 pl to a few microlitres, for example. When a technique for displacing the droplet over the surface is used, the droplet preferably has a volume of 10 pl to 10 μl. The present invention therefore makes it possible to carry out chemical or biochemical reactions in wall-less reactors having a small volume.

According to the invention, the surface onto which the droplet is deposited is preferably a surface that allows the formation of a droplet of ionic liquid without the latter spreading out too much, in particular in order to prevent contiguous droplets, that are not intended to coalesce, from touching one another (unwanted contamination between droplets deposited onto the surface). It may, for example, be a surface of silica, a glass surface, a Teflon surface, etc. It is in fact the surface on which the chemical or biochemical reaction is carried out using the droplet microreactor of the present invention. It may be any surface suitable for fabricating a lab-on-chip, and preferably compatible with the ionic liquids. The material of the surface is therefore preferably compatible with the droplet format and, where appropriate, with the chosen technique for displacing the droplet(s). If a displacement technique is used, a preferred surface, for example of a lab-on-chip, is of course a surface that exhibits little adhesion with the ionic liquid(s) used, for example a hydroplethobic surface or a surface rendered hydroplethobic, for example made of Teflon.

The surface may have one or more cavity or cavities (hollow(s)) provided so as to receive the droplet(s); one or more projection(s); it may also be a planar surface without bumps; or else a combination of hollows and/or projections and/or planar surface. When an electrowetting displacement technique is used, the surface may be equipped with a conducting wire (counter electrode) that makes it possible to polarize the droplet so as to displace it as described below.

This surface may be that of a lab-on-chip known to those skilled in the art, covered or not covered with a cap.

The presence of a cap covering the droplet(s) and intended to prevent evaporation of the ionic liquid is advantageously not obligatory. However, it may be required it the chemical reaction carried out requires specific conditions, for example an inert atmosphere, an argon stream, or suctioning of toxic volatile products.

According to the invention, a first droplet of an ionic liquid and a second droplet of an ionic liquid can be deposited onto a surface, for example of a lab-on-chip. According to the invention, the expression “a first droplet of an ionic liquid and a second droplet of an ionic liquid” is intended to mean that at least two droplets that are identical or different, either by virtue of the nature of the ionic liquid or by virtue of the nature of the reagent(s) introduced into the ionic liquid, are deposited onto said surface. The present description applies, of course, independently to each of the droplets deposited onto said surface.

In the method of the present invention, regardless of the embodiment, it is possible to deposit, for example, 1, 2, 3, 4, 5, . . . to 1000 droplets or more onto the same surface, these droplets being identical or different by virtue of their volume and/or by virtue of the nature of the ionic liquid and/or by virtue of the nature of the reagents introduced into the ionic liquid. The present invention therefore exhibits a specific advantage, in particular by virtue of the ease with which it is implemented, for carrying out, on the same lab-on-chip, chemical and/or biochemical reactions in parallel, for example multiparametric reactions, for example on a sample to be analysed.

In the implementation of the method of the invention, a first droplet and a second droplet can be brought together. According to the invention, the expression “the first and the second droplets are brought together” is intended to mean that at least two droplets deposited onto the surface can be brought together, in particular so as to mix them and/or to mix their content, for example the first and second reagents. The term “first and second reagents” is intended to mean at least two reagents, it being possible for each of the droplets to comprise one or more reagents, it being possible for each of the droplets to consist of a functionalized or nonfunctionalized ionic liquid.

The bringing together of the two droplets, or coalescence, can therefore make it possible to initiate the chemical or biochemical reaction(s) or simply to carry out a mixing of the reagents and/or ionic liquids. For example, if one of the droplets comprises a task-specific ionic liquid and the other a matrix ionic liquid and a reagent, the bringing together, or bringing into contact, of these droplets of ionic liquid makes it possible to carry out the desired chemical reactions between the reagent and the function carried by the ionic liquid.

The bringing together of several droplets can be carried out simultaneously or successively. Specifically, firstly, two or more droplets can be brought together to form a single droplet so as to chemically react their content when they are mixed. Then, secondly, a third droplet or more can be added to the mixture of the previous two so as to carry out mixing or another chemical or biochemical reaction, and so on. Thus, a series of chemical and/or biochemical reactions can be carried out very readily, by simply bringing droplets together, by virtue of the present invention, for example on a lab-on-chip.

The implementation of the present invention can consist, according to a first example, of the succession of the following steps, as illustrated schematically in the attached FIG. 1:
-i- a first droplet of ionic liquid (LI---A) consisting of an ionic liquid, or of an onium salt, functionalized with a function A that may or may not be capable of reacting with a reagent B, is deposited onto a surface, for example in a reaction chamber of a lab-on-chip, and
-ii- a second droplet of a matrix ionic liquid containing a reagent B is deposited onto this surface,
-iii- the first and the second droplets are brought together, for example by means of a displacement technique such as those mentioned above, and
-iv- after a suitable period of chemical reaction between the function A and the reagent B, a droplet (LI---C) is obtained in which the ionic liquid is functionalized with the product (C) of the reaction A+B.

“---” indicates a chemical bond between the ionic liquid and the function or the molecule that functionalizes the ionic liquid. It may, for example, be a covalent bond, etc.

It is possible to carry out other chemical reactions after fusions with other droplets of ionic liquid containing other reagents.

In a second example (not represented), the two droplets of ionic liquid are matrix ionic liquids, each of the droplets comprises one of the reagents A and B, and the bringing into contact (coalescence) of these two droplets of LI makes it possible to carry out mixing of the reagents A and B in the droplet of LI formed from the two droplets brought together, or a reaction between the reagents A and B. In this example, the droplets may not be functional ionic liquids, but only matrices. In the latter case, the reagents are simply in solution in these matrices, which play the role of a solvent.

The implementation of the method of the invention can also consist, according to a third example, of the succession of the following steps, in addition to steps -i- to -iv- mentioned above, as illustrated schematically in the attached FIG. 2:
-v- a third droplet of an ionic liquid containing a reagent D is deposited onto this surface (at the same time as the deposition of the first two in steps -i- and -ii- or after step -ii- or -iv-);
-vi- the droplet of the previous step -iv- is brought together with the droplet of ionic liquid, for example by means of a displacement technique such as those mentioned above, and
-v- after a suitable period of chemical reaction between the product C and the reagent D, a droplet (LI---E) is obtained, in which the ionic liquid is functionalized with the product (E) of the reaction C+D.

In a fourth example, in which the ionic liquids are all matrix ionic liquids, a single droplet comprising a mixture X+Y+Z is obtained by bringing together three droplets of ionic liquids each comprising one of the reagents X, Y and Z.

The present invention may also consist, according to a fifth example, of the implementation of a method for preparing a molecule M fixed on an initial function F₀, linked, in the droplet of ionic liquid, optionally by means of an arm L, in particular an alkyl group containing from 1 to 20 carbon atoms, to an ionic entity Y⁺—, which is part of the cation A₂⁺ of the functionalized salt A₂⁺X₂⁻ used, and/or Y⁻—, which is part of the anion X₂⁻ of the functionalized salt A₂⁺X₂⁻ used, the cation being in the form Y⁺-L-F₀ and/or the anion being in the form Y⁻-(L)ₖ-F₀, k being equal to 0 or 1, which method comprises the following steps, written based on the definitions of the ionic liquids provided above:
a first addition of a reagent B₁ in a droplet of ionic liquid of composition mentioned above and reaction between said function F₀ and the reagent B₁, resulting in a function F₁, linked to the ionic entity which is part of the cation A₂⁺ of the functionalized salt A₂⁺X₂⁻, and/or to the ionic entity Y⁻—, which is part of the anion X₂⁻ of the functionalized salt A₂⁺X₂⁻, according to one of the following reaction schemes:

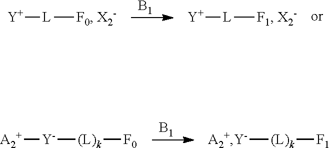

n−1 successive additions of reagents Bᵢ (as desired by means of a droplet of matrix ionic liquid, of solid reagents, of liquid reagents, or of a droplet of aqueous solution of Bᵢ) in the droplet of ionic liquid of composition mentioned above, 1<i≦n, n ranging from 2 to 10, allowing, at each addition, reaction between the reagent Bᵢ and a function Fᵢ₋₁, resulting in a function Fᵢ being obtained, the (n−1)^{th} addition of the reagent Bₙ on the function Fₙ₋₁ resulting in the function Fₙ being obtained, it being possible for the n−1 additions to be represented according to one of the following reaction schemes:

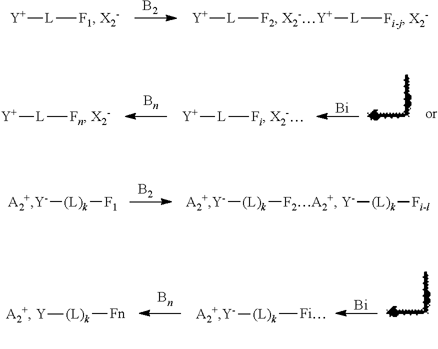

cleavage of the function Fₙ, linked to the ionic entity Y⁺- or Y⁻—, respectively, of the cation A₂⁺ and/or the anion X₂⁻, making it possible to recover, on the one hand, the functionalized salt A₂⁺X₂⁻ in the form Y⁺-L-F₀, X₂⁻ or A₂⁺, Y⁻-(L)ₖ-F₀, in solution in the ionic liquid A₁⁺X₁⁻, or in the form Y⁺-L-F′₀, X₂⁻ or A₂⁺, Y⁻-(L)ₖ-F′₀, in which F′₀ represents a function different from F₀,
and, on the other hand, the molecule M,
according to one of the following reaction schemes:

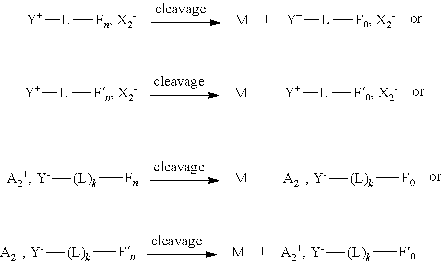

The reagents B₀ to Rₙ can be provided successively by means of a droplet of matrix ionic liquid fused to the droplet of functionalized ionic liquid. Molecule M is recovered at the end of the method of preparation carried out. Document [10] describes this type of protocol that can be used in the present invention.

In a sixth example, droplets of ionic liquids containing supported reagents can be fused, resulting, in the end, in a multisalt in solution in a matrix LI. It is then possible to return to the previous example and to react nonsupported reagents by means of fusion with droplets of matrix ionic liquids containing these reagents.

Thus, by virtue of successive coalescences of droplets according to the method of the invention, it is possible to successively carry out mixings and reactions, in matrix ionic liquids or with functionalized ionic liquids so as to carry out a very large number of types of chemical and biochemical reactions, in the same manner as in a conventional reactor.

There is therefore an infinite number of possibilities of series of steps in accordance with the present invention.

In these series, the matrix or functionalized ionic liquids used in the various reactions may be identical or different. Thus, according to the invention, said at least a first ionic liquid and said at least a second ionic liquid are independently chosen from a functionalized or nonfunctionalized ionic liquid. The first ionic liquid can therefore comprise, in addition to the functionalized ionic liquid, a nonfunctionalized ionic liquid, or alternatively in addition to the nonfunctionalized ionic liquid, a functionalized ionic liquid. Similarly, and independently, the second ionic liquid can comprise, in addition to the functionalized ionic liquid, a nonfunctionalized ionic liquid, or alternatively, in addition to the nonfunctionalized ionic liquid, a functionalized ionic liquid. Of course, the first droplet and the second droplet may be identical or different and may independently have volumes as indicated above.

The step consisting in chemically or biochemically reacting the reagent or reagents with one another or with the function carried by an ionic liquid of a droplet is carried out like any chemical or biochemical reaction step in a conventional reactor of the prior art, i.e. a walled reactor, apart from the fact that it is carried out in the droplet microreactor of the present invention, i.e. in the droplet of functionalized or nonfunctionalized ionic liquid.

According to the invention, the reaction may be any chemical or biochemical reaction. By way of example of reactions that can be carried out in the microreactor of the present invention, mention may be made of the following reactions:
Combinatorial chemistry and synthesis reactions on a soluble support, for instance those described in document [11].
Enzymatic reactions; mention may, for example, be made of reactions using lipases, such as those described in document [12].
Catalytic reactions; mention may, for example, be made of olefin metathesis, such as that described in document [13].
Dangerous reactions; mention may, for example, be made of reactions involving azides, as described′ in document [14].
Electrochemical reactions; mention may, for example, be made of cathodic cleavages of bonds, as described in document [15].
Heterogenization of catalysts and homogeneous catalyses.
Chemical or biochemical reaction optimizations.
Parallel syntheses.
Convergent syntheses.
Immobilizations on ionic liquids of chemical or biological molecules capable of being detected (target molecules) or of detecting (probe molecules), for example proteins, enzymes, nucleic acids (DNA and RNA), glycoproteins, lipids, etc.

In this reaction step, the operating conditions suitable for carrying out the chemical or biochemical reaction in question in a conventional reactor of the prior art are therefore implemented in the present invention in a droplet of ionic liquid. For example, each of the droplets that forms a microreactor can be heated so as to allow conventional organic chemistry reactions, for example up to 200° C. or more, due to the non-volatility of the ionic liquids. The chemical reactions carried out in the ionic liquids can be carried out at ambient temperature, but also at high temperatures.

The product(s) obtained during or after the chemical reaction(s) carried out in the droplet of ionic liquid may then be detected or quantified, either directly inside the lab-on-chip, for example by colorimetric or electrochemical detection or any other suitable means of detection known to those skilled in the art, or else outside the lab-on-chip, for example by high performance chromatography (HPLC) techniques, gas chromatograpy (GC) techniques, by techniques of spectroscopic analysis, by nuclear magnetic resonance (NMR), by infrared (IR), by ultraviolet (UV) in the visible range, by mass spectrometry (MS), by liquid chromatography coupled to mass spectrometry (LC/MS), by colorimetry, or by any other suitable analytical technique known to those skilled in the art for detecting the molecules to be analysed.

The analyses can be carried out directly in the droplet (for example by NMR, HPLC or another technique such as those mentioned above), or after release of the product of the reaction linked to the ionic liquid, by cleavage (see Example 1), and/or extraction and/or purification of the product(s) derived from the reaction carried out in the droplet of ionic liquid. This extraction can be carried out, for example, by the technique described in document [10].

According to the invention, regardless of the method used, it may also comprise a step consisting in displacing the droplet(s) of ionic liquid over the surface.

This displacement of the droplet(s) may have various objectives, among which mention may, for example, be made of that of bringing together two or more droplets of ionic liquid deposited onto the surface in the abovementioned applications of mixing(s) and chemical or biochemical reaction(s) between the droplets and their content; but also that of displacing a droplet of ionic liquid from one reaction zone of a lab-on-chip to another reaction zone of said lab, or else from a reaction zone of a lab-on-chip to a detection zone of said lab.

The displacement of the droplet microreactors of the present invention can be carried out by any technique known to those skilled in the art for displacing a droplet over a surface.

Advantageously, according to the invention, it is a displacement technique chosen from:
Mechanical displacement, for example by vibration, by capillary action, by means of a button or else by transport on a mobile support. An example of transport on a mobile support that can be used in the present invention is a “conveyor belt” as described, for example, in document [16].
Electrostatic displacement, for example by electrowetting. The technique for displacement of the droplets of ionic liquid by electrowetting was discovered in the context of the present invention. Specifically, during their research, the inventors of the present invention were the first to demonstrate the property that ionic liquids have of being able to be displaced over a surface, in the form of a droplet, by electrowetting. This technique is particularly advantageous in the implementation of the method of the invention, especially in “lab-on-chip” applications. It is, for example, possible to use the electrowetting-on-dielectric described in document [5], where the forces used are electrostatic forces. The droplet lies on a network of electrodes, from which it is insulated by a dielectric layer and a hydrophobic layer. When the electrode in proximity to the droplet is activated, since the dielectric layer and the hydrophobic layer between the activated electrode and the droplet, that is continually polarized by a counterelectrode, act as a capacitance, the electrostatic charge effects induce displacement of the droplet over the activated electrode. The counterelectrode is essential to displacement by electrowetting, it maintains an electric contact with the droplet during its displacement. This counterelectrode can be either a catenary (Ca) as described in [5], or a buried wire, or a planar electrode on the cap of contained systems. The electrodes can be produced by coating with a metal layer, for example a metal chosen from Au, Al, ITO, Pt, Cr and Cu, or by photolithography. The substrate is then coated with a dielectric layer, for example of Si₃N₄ or of SiO₂. Finally, coating with a hydrophobic layer is carried out, for instance spin-coating with Teflon. By virtue of the electrowetting technique, it is possible to displace the droplets of ionic liquids so that they become closer and closer, and, optionally, to bring them together so as to mix them, in order to carry out complex protocols. Document [5] gives examples of uses of series of adjacent electrodes for manipulating a droplet in a plane, that can be used in the present invention. Displacements of this type can be used, for example, in biochemical-, chemical- or biological-test devices in the medical field, environmental monitoring, quality control, etc.
Displacement by dielectric forces. This technique consists in manipulating an interface between two immiscible fluids. Document [17] describes, for example, an electrically controlled device for dielectric liquid displacement that can be used in the present invention. A droplet of liquid is placed between two planes comprising electrode couples. The droplet of liquid exhibits a permittivity greater than its environment defined by the space between the two planes comprising the electrodes. The displacement is electrically controlled by applying electric voltages to the electrocouples. A variant of this technique, that can be used in the present invention, is described in document [18].
Displacement by thermal gradient or by electrocapillarity, for example by the technique described in document [19]. The technique consists in immersing a droplet of ionic liquid in a thermal gradient. This results in fluid circulation at the interface of the droplet by virtue of the Marangonie effect. This fluid circulation causes the droplet to be displaced.
Pressure-wave or acoustic-wave displacement, for example by the technique described in document [20]. The technique consists in propagating acoustic waves over a hydrophobic surface. The wave disturbs the wetting of the droplet, and causes its displacement.

The present invention makes it possible to carry out chemical or biochemical reactions in wall-less reactors of small volume. In addition, the task-specific ionic liquids make it possible to carry out chemical reactions with the same reactivity as in solution. Furthermore, the reactions can be monitored and the reaction products can be readily purified, for example after cleavage.

With the droplet microreactor of the present invention, there is no blocking of channels, there is no load loss in hydrodynamic mode, for example when syringe-pumps or pumps are used, and there are no dead volumes as there are with the microreactors of the prior art.

In addition, unlike with channels, with the present invention, there is no diffusion problem. The reactions remain at constant concentration and individualized.

Furthermore, the microsystem of the present invention is a microsystem that is inexpensive to fabricate and compatible with an aggressive chemical environment, in particular due to the solvents used, the working temperatures, the pressures, etc.

Other characteristics and advantages of the invention will further emerge on reading the examples which follow, given by way of nonlimiting illustration with reference to the attached figures.

### BRIEF DESCRIPTION OF THE FIGURES

FIG. 1: Schematic representation of a chemical and/or biochemical reaction for producing a product C in a droplet microreactor, carried out by means of the method of the present invention by bringing together a droplet of ionic liquid functionalized with a function A (LI---A) and a droplet of matrix ionic liquid comprising the reagent B.

FIG. 2: Schematic representation of a chemical and/or biochemical reaction for producing a product E in a droplet microreactor, carried out by means of the method of the present invention by bringing together a droplet of functionalized ionic liquid (LI---A) and a droplet of matrix ionic liquid comprising the reagent B, so as to form the product C immobilized on the ionic liquid (LI---C), and then by bringing LI---C together with a droplet of matrix ionic liquid comprising the reagent D.

FIG. 3: Schematic representation of the displacement of a droplet of ionic liquid by electrowetting so as to carry out the method of the present invention.

FIG. 4: Graph representing the plot of a chromatogram (A (absorbance)=f(time in minutes)) obtained on a droplet of task-specific ionic liquid before carrying out a chemical or biochemical reaction according to the method of the present invention.

FIG. 5: Graph representing the plot of a chromatogram (A=f(time in minutes)) obtained on the droplet of task-specific ionic liquid of FIG. 4, but after chemical or biochemical reaction and washing according to the method of the present invention.

FIGS. 6A-C: Scheme of a device for displacing droplets of ionic liquid by electrowetting so as to carry out the method of the present invention when it comprises a displacement step.

FIG. 7: Graph representing the plot of a chromatogram (A=(f(time in minutes)) obtained on the droplet of task-specific ionic liquid at the time of a reaction to detach a reaction product fixed on an ionic liquid after carrying out a method of the invention.

FIG. 8: Spectrometry spectrum obtained in Example 4 (intensity=f(m/z)).

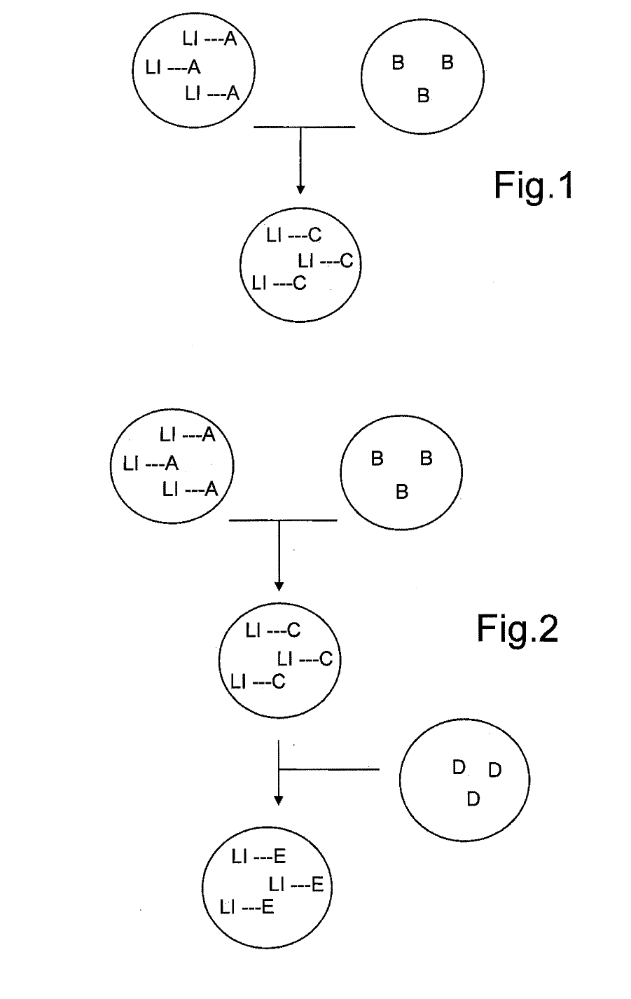

### EXAMPLES

### Example 1

### Implementation of the Method of the Present Invention with Displacement of the Droplets so as to Carry Out a Series of Several Chemical Reactions: In the Case of the Grieco Reaction

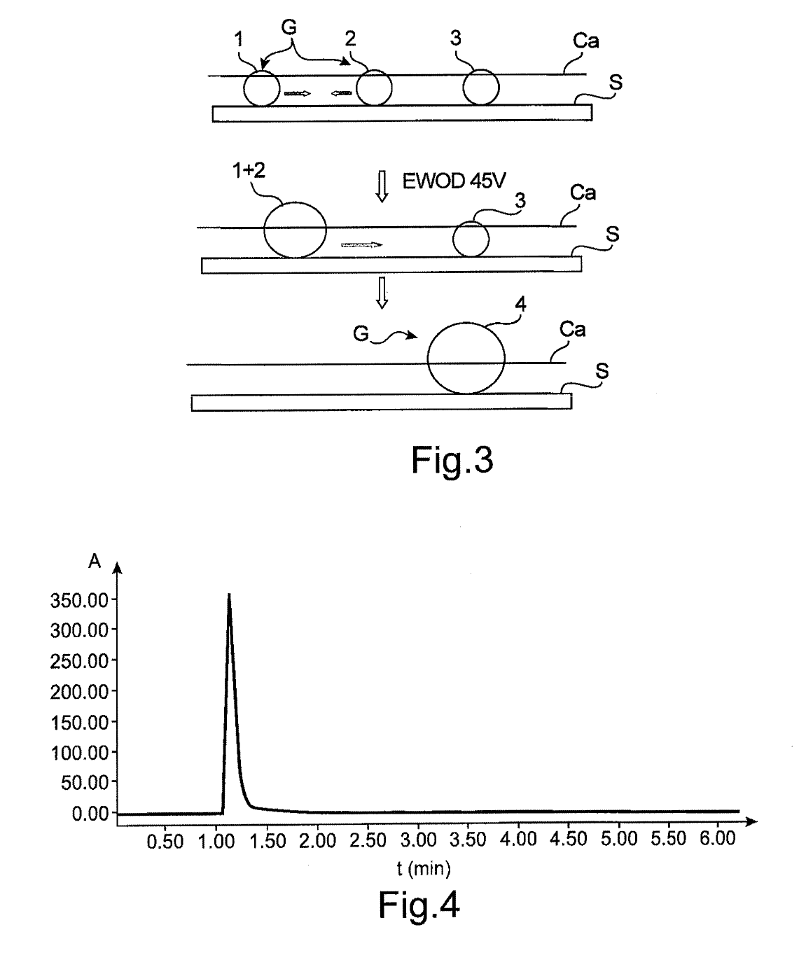

Three droplets of ionic liquids, each having a volume of 0.5 μl, are deposited onto a Teflon-coated surface of the reaction chamber described in document [16] and represented schematically in the attached FIGS. 3 and 6.

The droplets used in this example have the following composition:
droplet 1: droplet of 4-aminobenzoic acid supported on ammonium salt 1 (see reaction scheme below) in a 1 M solution in [tmba][NTf₂];
droplet 2: droplet of 2 equivalents of 4-nitrobenzaldehyde and 1.2 equivalents of TFA in a 0.5 M solution in [tmba][NTf_{z}]; and
droplet 3: droplet of 10 equivalents of indene in a 1 M solution in [tmba][NTf_{z}].

The droplet displacement technique used in this example is an electrowetting displacement technique which operates as represented schematically in FIG. 6 (a single droplet is represented in FIG. 6): the support (S) is structured so as to comprise a network of electrodes (E), a dielectric layer (D), a hydrophobic layer (H) and connection means (Co) connected to a power source (V). The droplets (G) lie on the network of electrodes (FIG. 6A), from which they are insulated by the dielectric layer and the hydrophobic layer.

When one of the electrodes, in proximity to the droplet, is activated, the dielectric layer and the hydrophobic layer between the activated electrode and the droplet under voltage acts as a capacitance, the surface becomes charged, and since the droplet continually polarized by a counterelectrode acts as a capacitance, the electrostatic charge effects induce the displacement of the droplet over the activated electrode. The counterelectrode is essential to the displacement by electrowetting, it maintains an electrical contact with the droplet during its displacement. This counterelectrode is in this case a catenary (Ca). The electrodes are produced by coating with a layer of gold, by photolithography. The substrate is then coated with a layer of SiO₂. Finally, a layer of Teflon is deposited by spin-coating.

The droplet is electrostatically attracted on the surface of this electrode (FIG. 6B). Thus, it is possible to displace the droplets of ionic liquids closer and closer together and also to mix them by selectively activating one or other of the electrodes of the electrode network. A catenary (Ca), placed on the support, makes it possible to polarize the droplet.

In this example, the abovementioned droplets are displaced so as to be successively brought together. FIG. 3 is a schematic representation of the protocol used: the 1st droplet (1) is displaced towards the 2nd (2), the 2nd towards the 1st and, after these two droplets have been brought together, the droplet (1+2) formed by mixing them is displaced towards the 3rd (3) droplet so as to form a droplet (4).

After fusion of the three droplets, the mixture which is obtained is incubated at ambient temperature for 15 minutes. The Grieco reaction is then complete.

After reaction in the droplet, the droplet (1.5 μl) is recovered in an Eppendorf tube and washed several times with ether (3×20 μl) in order to extract from the ionic liquid the excess products or alternatively the by-products. The ether solubilizes these products, but is not miscible with the ionic liquid chosen. The ionic liquid is then freed of the excess products or alternatively the by-products.

The chemical reaction carried out in this example is summarized by the reaction scheme below, in which [btma][NTf₂] represents the matrix ionic liquid used, and in which TFA represents trifluoroacetic acid.

The product 2 is cleaved from the support after an overnight incubation at ambient temperature in the presence of a 7N solution of NH₃ in methanol. Thus, on the reaction scheme below, the treatment X comprises the following successive steps:

1) washing with ether;
2) NH₃/MeOH, at ambient temperature (T°A); and
3) extraction with ether after evaporation of the methanol.

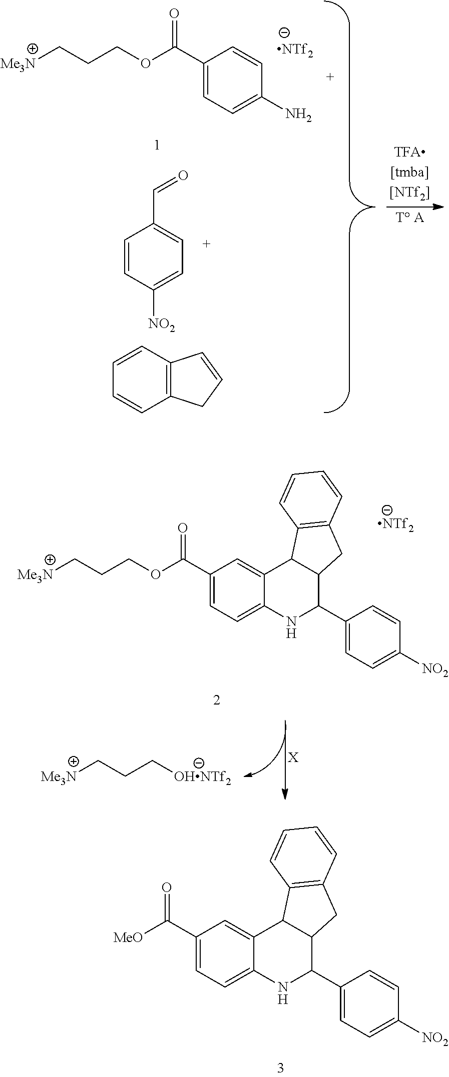

A reverse-phase HPLC analysis of the reaction demonstrates the appearance of the final product with a retention time that is different from that observed for the starting functionalized salt.

The HPLC analysis conditions used were as follows:
Column: C₁₈ Nova-Pak (registered trade mark), 3.9×150 mm column, part No. WAT 086344, Waters (trade mark)
Conditions:
   CH₃CN/H₂O: 2:1
   CH₃CN HPLC (Carlo Erba—(trade mark))
   Solution of H₂O composed of 20 mmol of ammonium acetate and 1% of acetic′acid
   λ=254 nm
   Flow rate=1.5 ml/minute
   Pressure: 9.58×10⁵ to 10.07×10⁵ Pa (1390-1460 Psi)
   Column temperature: 30° C.

FIG. 4 is the plot of the chromatogram obtained on the droplet of task-specific ionic liquid (1) before chemical reaction is carried out.

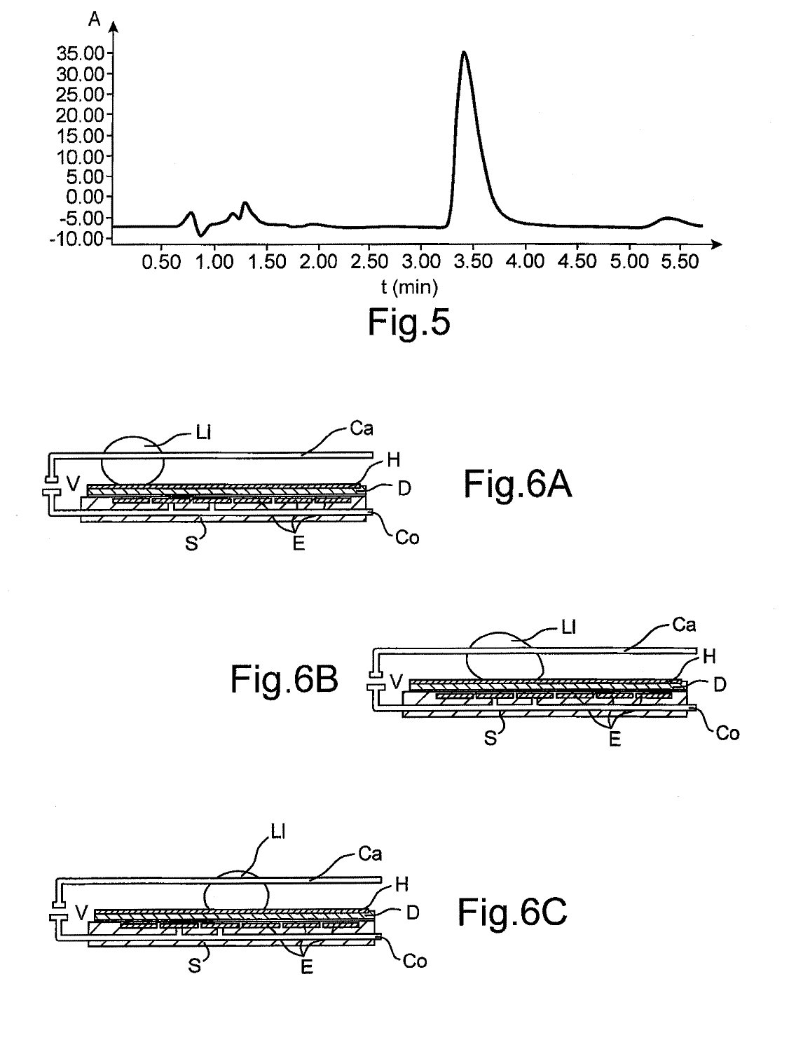

FIG. 5 is the plot of the chromatogram obtained on the droplet of ionic liquid (4) after chemical reaction and washing.

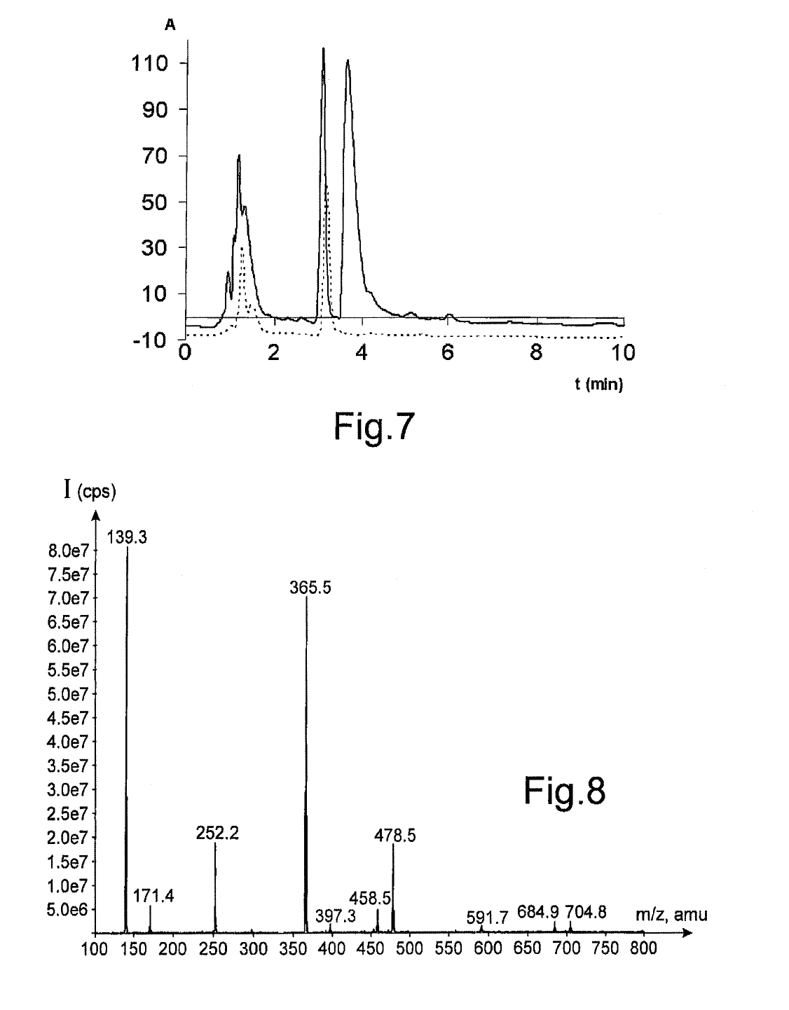

FIG. 7 is the plot of the chromatogram that makes it possible to follow the cleavage carried out by means of the treatment X enabling release of the reaction product. The disappearance of the product 2, the retention time of which is 3.65 min, and the appearance of 3 at 3.06 min are observed. On this figure, as a continuous line, the ploy obtained after three hours of contact with NH₃/MeOH: mixture of starting product and of trans-esterified product; as a discontinuous line, the trans-esterification reaction completed (and therefore detachment from the ionic liquid) after overnight reaction (12 hours), the starting product is no longer detectable.

### Example 2

### Detritylation Reaction Carried Out According to the Method of the Present Invention

Two droplets of matrix ionic liquid ([btma][NTf₂]), each of 0.5 μl, are deposited on the Teflon-coated surface of the reaction chamber described in document [16].

Each of the droplets contains a reagent: droplet No. 1 contains a tritylated thymidine base and droplet No. 2 contains dichloroacetic acid.

Droplet No. 1 is made to converge towards the other droplet using the electrowetting technique. The voltage applied is 45 V.

After fusion of the two droplets, the mixture is incubated at ambient temperature for 5 minutes. An orangey coloration of the droplet demonstrates the formation of the desired product.

The chemical reaction carried out is the following:

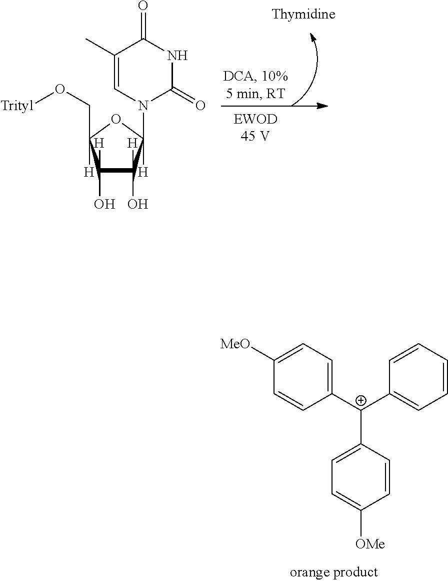

In which DCA is dichloroacetic acid and EWOD represents the displacement by electrowetting.

### Example 3

### Implementation of an Enzymatic Reaction

A first reaction mixture is prepared as follows: 50 mM citrate-phosphate buffer, pH 6.5 (10 ml), o-phenylene-diamine (OPD, 20 mg) and aqueous hydrogen peroxide (4 μl).

A droplet of this mixture, 0.5 μl in volume, is dissolved in matrix ionic liquid ([btma][NTf₂]) (0.5 μl).

A second reaction mixture is prepared as follows: matrix ionic liquid ([btma][NTf₂]) (0.9 μl) and horseradish peroxidase (0.1 μl at 20 μm).

A droplet (0.5 μl) of each of the mixtures is deposited onto the Teflon-coated surface of the reaction chamber used in Examples 1 and 2 above.

Droplet No. 2 is made to converge towards the other droplet using the electrowetting technique. The voltage applied is 45 V.

After fusion of the two droplets, the mixture is incubated at ambient temperature for 20 minutes.

A brown coloration characteristic of the enzymatic reaction for forming 2,3-diaminophenazine is observed.

### Example 4

### Implementation of the Method of the Present Invention with Displacement of the Droplets so as to Carry Out a Reduction Reaction

Two droplets of ionic liquids, each 0.3 μl in volume, are deposited onto a Teflon-coated surface of the reaction chamber described in document [16] and represented schematically in the attached FIGS. 3 and 6.

The droplets used in this example have the following composition:
droplet 1: droplet of aldehyde supported on an ammonium salt 4 (see reaction scheme below) in solution [0.5 M] in [bmim][BF₄];
droplet 2: droplet of BH₃.pyridine [10 equivalents] in [bmim][BF₄].

One of the droplets is then made to converge towards the other by electrowetting, by applying a voltage of 55 V.

After fusion of the droplets, the mixture obtained is incubated at ambient temperature (18-25° C.) for 2 hours.

After reaction in the droplet, the latter (0.6 μl) is recovered in an Eppendorf (registered trade mark) tube and washed several times with ether (3×20 μl) in order to extract from the ionic liquid the excess products or alternatively the by-products. The ether solubilizes these products, but is not miscible with the ionic liquid chosen.

The mixture is then injected into positive-mode (electrospray) mass spectrometry. The spectrum represented in the attached FIG. 8 is thus obtained, showing, at 252.2 uma, the molecular ion corresponding to the alcohol 5 derived from the reduction of the aldehyde. The peaks at 139.3, 365.5 and 478.5 correspond, respectively, to the bmim⁺. and [2bmim, BF₄⁻]⁺. ions and to the adduct [alcohol 5, bmim, BF₄⁻]⁺..

### Reaction Scheme of Example 4

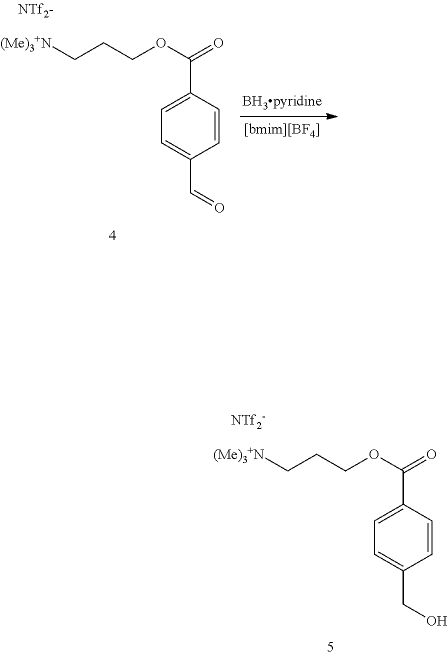

### REFERENCE LISTING

[1] P. D. I. Fletcher et al, Lab on a chip, 2003, 309-333.
[2] K. Jahnisch et al, Angew. Chem. Int. Ed., 2004, 43, 406-446.
[3] S. J. Haswell and P. Watts, GB-A-2 387 382.
[4] Tomohiro Taniguchi, Toru Torii, Toshiro Higuchi, Lab on a chip, 2002, 2, 19-23.
[5] M. G. Pollack et al, Microtas 2003, vol. 1, p. 619.
[6] Linstrom, chem rev, 2002, 102, 2751-2772.
[7] S. R. Wilson and A Czarnik, “Combinatorial Chemistry: Synthesis and Application”; John Wiley and Sons New York, 1997;
[8] D. G. Gravet and K. D. Janda, Chem. Rev., 1997, 97, 489-510.
[9] T. Welton, Chemical Reviews, 1999, 99(8), 2071-2083.
[10] S. Gmouth, M. Vaultier, FR-A-2 845 084 (WO 2004029004).
[11] Angew. Chem. Int. Ed. Engl., 1996, 35, 2288-2337 (Balkenhohl et al, Revue).
[12] “Journal of Molecular Catalysts A: chemical, 2004, 214, 1, p 113-119 (Vaultier et al)
[13] JACS, 2003, 125, 9248-9249 (J. C. Guillemin et al).
[14] “Advanced Organic Chemistry, Reactions, Mechanisms and structures, Jerry March, Fourth edition, Wiley New York, 1992.
[15] “Actualité chimique” [Chemical news], August-September 1998, p 442 (J. Simonet).
[16] Y. Fouillet, R. Charles, O. Constantin, H Jeanson, WO-A-02/061438 (FR-A-2 841 063).
[17] J. P. Pesant, M. Hareng, FR-A-2 548 431.
[18] J. A. Schwartz, J; V. Vykoukal and P. R. C. Gascoyne, “Droplet-based Chemistry on a programmable microchip”, Lab-on-a-chip, 2004, 4.
[19] Yarin A. L., Liu W., Reneker D. H. J. Appl. Phys. Vol. 91, No. 7, 4751-4760 (2002).
[20] A. Wixforth, J. Scriba, C. Gauer; MST-NEWS, 5-2002.

1. Microreactor characterized in that it consists of a droplet comprising at least one ionic liquid, wherein the limits of the microreactor are defined by the interface of the at least one ionic liquid and the ambient environment and with a support on which the droplet is deposited, and wherein the droplet has a volume of 1 pl to 10 μl, in which the at least one ionic liquid is a mixture comprising an ionic liquid and an organic solvent different from said ionic liquid, and wherein said ionic liquid is in liquid form at ambient temperature.

2. Microreactor according to claim 1, in which the at least one ionic liquid comprises a solvent.

3. Microreactor according to claim 1, in which the at least one ionic liquid comprises at least one reagent.

4. Microreactor according to claim 1, in which said at least one ionic liquid is a functionalized or nonfunctionalized ionic liquid.

5. Microreactor according to claim 4, comprising:
in addition to the functionalized ionic liquid, a nonfunctionalized ionic liquid, or
in addition to the nonfunctionalized ionic liquid, a functionalized ionic liquid.

6. Microreactor according to claim 1, in which the at least one ionic liquid is chosen from an ammonium salt, a phosphonium salt, an imidazolium salt, an onium salt, or a mixture of these salts.

7. Method for carrying out a chemical or biochemical reaction, comprising the following steps:
depositing a droplet of at least one ionic liquid is a mixture comprising an ionic liquid and an organic solvent different from said ionic liquid onto a surface,
introducing, into the at least one ionic liquid, before or after it is deposited in the form of a droplet, at least one chemical or biochemical reagent,
chemically or biochemically reacting, in said droplet, the reagent with the ionic liquid and/or the reagents with one another,
wherein said ionic liquid is in liquid form at ambient temperature.

8. Method according to claim 7, in which said at least one ionic liquid comprises a functionalized or nonfunctionalized ionic liquid.

9. Method according to claim 8, in which the droplet comprises:
in addition to the functionalized ionic liquid, a nonfunctionalized ionic liquid, or
in addition to the nonfunctionalized ionic liquid, a functionalized ionic liquid.

10. Method according to claim 7, in which the droplet has a volume of 1 pl to 10 μl.

11. Method according to claim 7, in which the at least one ionic liquid is chosen from an ammonium salt, a phosphonium salt, an imidazolium salt and an onium salt, or a mixture of these salts.

12. Method according to claim 7, in which the droplet is deposited onto the surface by means of a technique chosen from the group comprising manual deposition, deposition by means of an automated or nonautomated droplet dispenser, or deposition by fractionation of a larger droplet deposited onto the surface.

13. Method according to claim 7, in which the surface is a surface of a lab-on-chip.

14. Method according to claim 7, in which the surface is a polytetrafluoroethylene surface.

15. Method according to claim 7, also comprising a step consisting in displacing the droplet of ionic liquid over said surface by means of a technique chosen from mechanical displacement, electrostatic displacement, thermal displacement or acoustic displacement.

16. Method for mixing droplets of ionic liquid, comprising the following steps:
depositing a first droplet of at least one first ionic liquid onto a surface, wherein the at least one first ionic liquid is a mixture comprising an ionic liquid and an organic solvent different from said ionic liquid;
depositing a second droplet of at least one second ionic liquid onto said surface;
optionally, introducing into the first ionic liquid, before or after it is deposited in the form of a droplet onto the surface, at least a first chemical or biochemical reagent;
optionally, introducing into the second ionic liquid, before or after it is deposited in the form of a droplet onto the surface, at least a second chemical or biochemical reagent;
bringing by electrowetting the first droplet and the second droplet together so as to form a single droplet,
wherein said ionic liquid is in liquid form at ambient temperature, and wherein said first droplet is displaced over the surface by electrowetting.

17. Method for carrying out a chemical or biochemical reaction, comprising the following steps:
depositing a first droplet of at least one first ionic liquid onto a surface, wherein the at least one first ionic liquid is a mixture comprising an ionic liquid and an organic solvent different from said ionic liquid onto a surface;
depositing a second droplet of at least one second ionic liquid onto said surface;
introducing into the first ionic liquid, before or after it is deposited in the form of a droplet onto the surface, at least a first chemical or biochemical reagent;
introducing into the second ionic liquid, before or after it is deposited in the form of a droplet onto the surface, at least a second chemical or biochemical reagent;
bringing the first droplet and the second droplet together on said surface so as to form a single droplet; and
chemically or biochemically reacting, in the droplet formed by the first and the second droplets brought together, said first reagent with said second reagent, wherein said ionic liquid is in liquid form at ambient temperature, and wherein said first droplet is displaced over the surface by electrowetting.

18. Method according to claim 17, in which the at least one first ionic liquid comprises at least one reagent.

19. Method according to claim 17, in which said at least one first ionic liquid and said at least one second ionic liquid are independently chosen from a functionalized or nonfunctionalized ionic liquid.

20. Method according to claim 19, in which the at least one first ionic liquid comprises:
in addition to the functionalized ionic liquid, a nonfunctionalized ionic liquid, or
in addition to the nonfunctionalized ionic liquid, a functionalized ionic liquid.

21. Method according to claim 22, in which the at least one second ionic liquid comprises:
in addition to the functionalized ionic liquid, a nonfunctionalized ionic liquid, or
in addition to the nonfunctionalized ionic liquid, a functionalized ionic liquid.

22. Method according to claim 17, in which the first droplet and the second droplet are identical or different and each have a volume of 1 nl to 10 μl.

23. Method according to claim 17, in which the at least one first ionic liquid and the at least one second ionic liquid are each, independently of one another, chosen from an ammonium salt, a phosphonium salt, an imidazolium salt, an onium salt, or a mixture of these salts.

24. Method according to claim 17, in which the first and/or the second droplet is (are) deposited onto the surface by means of a technique chosen from the group comprising manual deposition, deposition by means of an automated or nonautomated droplet dispenser, and deposition by fractionation of a larger droplet deposited onto the surface.

25. Method according to claim 17, in which the surface is a surface of a lab-on-chip.

26. Method according to claim 17, in which the surface is a polytetrafluoroethylene surface.

27. Method according to claim 17, also comprising a step consisting in displacing the first droplet of ionic liquid and/or the second droplet of ionic liquid over the surface by means of a technique chosen from mechanical displacement, electrostatic displacement, thermal displacement or acoustic displacement.

28. Method according to claim 7, in which the chemical or biochemical reaction is a reaction chosen from the group consisting of a synthesis reaction, a reaction for the immobilization of chemical or biological molecules on the surface, an enzymatic reaction, a catalyst heterogenization reaction, a catalytic reaction, and an electrochemical reaction.

29. Lab-on-chip comprising at least one microreactor according to claim 1.

30. Method according to claim 18, in which the at least one first ionic liquid comprises at least one reagent.

31. Method according to claim 18, in which said at least one first ionic liquid and said at least one second ionic liquid are independently chosen from a functionalized or nonfunctionalized ionic liquid.

32. Method according to claim 31, in which the at least one first ionic liquid comprises:
in addition to the functionalized ionic liquid, a nonfunctionalized ionic liquid, or
in addition to the nonfunctionalized ionic liquid, a functionalized ionic liquid.

33. Method according to claim 31, in which the at least one second ionic liquid comprises:
in addition to the functionalized ionic liquid, a nonfunctionalized ionic liquid, or
in addition to the nonfunctionalized ionic liquid, a functionalized ionic liquid.

34. Method according to claim 18, in which the first droplet and the second droplet are identical or different and each have a volume of 1 nl to 10 μl.

35. Method according to claim 18, in which the at least one first and the at least one second ionic liquids are each, independently of one another, chosen from an ammonium salt, a phosphonium salt, an imidazolium salt, an onium salt, or a mixture of these salts.

36. Method according to claim 18, in which the at least one first and/or the at least one second droplet is (are) deposited onto the surface by means of a technique chosen from the group comprising manual deposition, deposition by means of an automated or nonautomated droplet dispenser, and deposition by fractionation of a larger droplet deposited onto the surface.

37. Method according to claim 18, in which the surface is a surface of a lab-on-chip.

38. Method according to claim 18, also comprising a step consisting in displacing the first droplet of ionic liquid and/or the second droplet of ionic liquid over the surface by means of a technique chosen from mechanical displacement, electrostatic displacement, thermal displacement or acoustic displacement.

39. Method according to claim 18, in which the chemical or biochemical reaction is a reaction chosen from the group consisting of a synthesis reaction, a reaction for the immobilization of chemical or biological molecules on the surface, an enzymatic reaction, a catalyst heterogenization reaction, a catalytic reaction, and an electrochemical reaction.

40. Microreactor characterized in that it consists of a droplet comprising at least one ionic liquid and an organic solvent different from said at least one ionic liquid, wherein said at least one ionic liquid is selected from the group consisting of:
1-butyl-3-methylimidazolium tetrafluoroborate [bmim][BF₄];
1-butyl-3-methylimidazolium hexafluorophosphate [bmim][PF₆];
1-butyl-3-methylimidazoliumbis(trifluoromethylsulphonyl)imide [bmim][NTf₂]; and
butyltrimethylammoniumbis(trifluoromethylsulphonyl)imide [btma][NTf₂],
wherein the limits of said microreactor are defined by the interface of the ionic liquid with the ambient environment and with a support on which the droplet is deposited.
